(19) United States
(12) Patent Application Publication
(10) Pub. No.: US 2007/0213558 A1
(43) Pub. Date: Sep. 13, 2007

(54) PROCESS FOR THE PREPARATION OF HYDROXY CARBOXYLIC ACID

(75) Inventors: Chaudhari Raghunath Vitthal, Pune (IN); Rajurkar Kalpendra Baburao, Pune (IN); Tonde Sunil Sopana, Pune (IN); Borole Yogesh Laxman, Pune (IN)

Correspondence Address: FULBRIGHT & JAWORSKI L.L.P., 600 CONGRESS AVE., SUITE 2400, AUSTIN, TX, 78701, US

(73) Assignee: Council of Scientific & Industrial Research, New Delhi (IN)

(21) Appl. No.: 11/535,008

(22) Filed: Sep. 25, 2006

(30) Foreign Application Priority Data
Mar. 10, 2006 (IN) 0655/DEL/2006

(51) Int. Cl. C07C 51/16; C07C 65/03

(52) U.S. Cl. 562/471; 562/525

(57) ABSTRACT

The present invention provides a three step process for the preparation of hydroxy carboxylic acid. The present invention involves the hydroformylation of enol ester (e.g. vinyl acetate) using a cobalt catalyst (e.g. Co₂(CO)₈), optionally a promoter or a ligand containing O, N, N—O, P, As or Sb atom/s, to obtain a mixture of 2-acetoxy carboxaldehyde (e.g. 2-acetoxy propanal) and 3-acetoxy carboxaldehyde (e.g. 3-acetoxy propanal), oxidizing the product acetoxy carboxyaldehydes to the corresponding acetoxy carboxylic acid, in presence or absence of a Gr. 8 metal catalyst, followed by hydrolyzation of the product acetoxy carboxylic acids to the corresponding desired hydroxy carboxylic acids, in presence of an acidic catalyst and separating the catalyst and reusing it for another hydrolysis step.

### BACKGROUND OF THE INVENTION

1. Field of the Invention

The present invention relates to a process for the preparation of hydroxy carboxylic acids. Particularly, the present invention relates the process wherein an enol acylate is hydroformylated in presence of carbon monoxide and hydrogen, a cobalt catalyst and optionally a promoter or a ligand containing O, N, N—O, P, As or Sb atom/s, to obtain a mixture of 2-acetoxy carboxaldehyde and 3-acetoxy carboxaldehyde, which on oxidation in presence of air or oxygen as oxidant, in presence or absence of a Gr. 8 metal catalyst gives a mixture of 2-acetoxy carboxylic acid and 3-acetoxy carboxylic acid, which on further hydrolysis in presence of water using an acidic catalyst gives a mixture of 2-hydroxy carboxylic acid and 3-hydroxy carboxylic acid, in which the required content of the individual carboxylic acid can be achieved by selecting a cobalt catalyst, a promoter and a set of reaction conditions. The process has a potential importance when applied to vinyl acetate. In preferred embodiment vinyl acetate is hydroformylated to give a mixture of 2-acetoxypropanaldehyde and 3-acetoxypropanaldehyde, which on oxidation produce a mixture of 2-acetoxypropanoic acid and 3-acetoxypropanoic acid, hydrolysis of which yield a mixture of 2-hydroxypropanoic acid and 3-acetoxypropanoic acid, which can be separated by fractional distillation to achieve the individual identities.

2. Description of Related Art

Hydroxy propionic acids viz. 2-hydroxy propionic acid (lactic acid) and 3-hydroxy propionic acid are important C₃ building blocks. Lactic acid is important commercially in polymer industry, baking industry, cheese industry, in dying wool, to make plasticizers for resin, etc. 3-hydroxy propionic acid is an important specialty chemical with wide ranging applications in pharmaceutical as well as biochemical industry.

Lactic acid has been produced industrially by fermentation of molasses, but the process is costly and inefficient, which produces large amount of byproducts, making product separation and purification expensive. Another commercial rout for lactic acid is hydrocyanation of acetaldehyde followed by hydrolysis of cyanohydrin with H₂SO₄. This rout is highly corrosive, consumes stoichometric amount of toxic HCN and H₂SO₄. Further the process uses expensive HCN and produces stoichometric amount of (NH₄)₂SO₄. U.S. Pat. No. 4,072,709 provides a process for the production of lactic acid in which, alpha-aceloxy-propanaldehyde formed by hydroformylation of vinyl acetate using Rh, Ir or Co catalysts is oxidized to alpha-aceloxy-propionic acid, which is further hydrolyzed to lactic acid. However, the process is aimed and applicable only for producing 2-hydroxy propionic acid and there is no method for producing 3-hydroxy propionic acid using such hydroformylation route. Also, the patent prefers Rh as a catalyst. U.S. Pat. No. 4,377,708 provides a process for hydrocarbonylation of vinyl acetate using CO and water as reactants with vinyl acetate using Pd-catalysts, which can produce lactic acid after hydrolysis. In the process, special precautions are taken for the stability of the catalyst, reactants and products. The process needs to maintain the concentration of water not more than 3 weight percent of the medium, so as to avoid hydrolysis of reactant vinyl acetate to acetic acid and acetaldehyde. Further, the process is applicable for producing a precursor for only 2-hydroxypropionic acid and not for 3-hydroxy propionic acid. European patent 0144118 provides a process for alkoxycarbonylation of enol esters with hydroxyl compounds using Pd, Rh and Ni catalysts and further hydrolysis of the products to only 2-hydroxy acids. However, the process operates at low concentration of hydroxyl compound (<10 times of enol ester), further the process doesn't provide catalyst separation method and reuse, showing inefficiency of the catalyst. Also, the process is not applicable for 3-hydroxy carboxylic acids.

Hydroformylation of vinyl carboxylate is known, in the art of literature, to give 2-carboxy propanal as a major product and 3-carboxy propanal as a minor product (if obtained any). The simplest vinyl carboxylate i.e. VAM on Rh-catalyzed hydroformylation produces almost selectively (90% regioselectivity) a branched aldehyde i.e. 2-acetoxy propanal. Hydroformylation of vinyl acetate has been reported in J. Am. Chem. Soc. 1949, 71, 3051, where 23% selectivity to 3-acetoxy propanal was achieved using Co₂(CO)₈ as a catalyst. However, the method used very high pressure of syn gas and also there is no report on the total three steps simultaneous synthesis of 2- and 3-hydroxy propionic acids.

As can be seen, the prior art processes suffer from several disadvantages such as use of costly and toxic chemicals, formation of large amount of byproducts, low catalyst activity, and catalyst and reactant stability. Further there is no efficient chemical method for preparation of 3-hydroxycarboxylic acid. It is therefore necessary to develop a process for preparation of 2-hydroxy carboxylic acids, 3-hydroxy carboxylic acid and simultaneous synthesis of 2- and 3-hydroxy carboxylic acid, which overcomes the drawbacks enumerated above.

### SUMMARY OF THE INVENTION

The main object of the present invention is to provide a process for preparation of hydroxy carboxylic acids, which obviates the drawbacks as detailed above.

Another object of the present invention is to provide an efficient process for preparation of 2-acetoxycarboxyaldehyde, 2-acetoxycarboxylic acid, 2-hydroxy carboxylic acid, 3-acetoxycarboxyaldehyde, 3-acetoxycarboxylic acid and/or 3-hydroxy carboxylic acid using cheaper catalysts at milder reaction conditions.

Still another object of the present invention is to provide a process for recycling the catalysts used for hydroformylation of enol acylates, oxidation of acetoxycarboxyaldehydes and hydrolysis of acetoxycarboxylic acids.

These and other related objects of the present invention are achieved by providing a process for preparation 2-hydroxy carboxylic acid and 3-hydroxy carboxylic acid, wherein an enol acylate is hydroformylated in presence of carbon monoxide and hydrogen, a recyclable cobalt catalyst and optionally a promoter or a ligand containing O, N, N—O, P, As or Sb atom/s, to obtain a mixture of 2-acetoxy carboxaldehyde and 3-acetoxy carboxaldehyde, which on oxidation in presence of air or oxygen as oxidant, in presence or absence of a homogeneous or heterogeneous recyclable Gr. 8 metal catalyst gives a mixture of 2-acetoxy carboxylic acid and 3-acetoxy carboxylic acid, which on further hydrolysis in presence of water using an acidic catalyst gives 2-hydroxy carboxylic acid and 3-hydroxy carboxylic acid.

Accordingly, the present invention provides a process for the preparation of hydroxy carboxylic acids, the said process comprising the steps of:
a) hydroformylating an enol acylate having formula R₂C═C(R)—O-Acyl, wherein R is H, alkyl or aryl group containing 1 to 15 carbon atoms free from any ethylenic or acetylenic unsaturation, and each R is the same or different, with a mixture of carbon monoxide and hydrogen, at a pressure in the range of 500 to 5000 psig, at a temperature in the range of 50 to 140° C., in presence of a catalyst containing cobalt, optionally in the presence of a promoter or a ligand containing O, N, N—O, P, As or Sb atom/s and an organic solvent to obtain a mixture of 2-acetoxy carboxaldehyde and 3-acetoxy carboxaldehyde,
a) oxidizing 2-acetoxy carboxaldehyde and 3-acetoxy carboxaldehyde either after their separation from the above said reaction mixture obtained in step (a) by distillation or solvent extraction, or as such in the reaction mixture without separation, in presence of air or O₂, optionally in the presence of an organic solvent, at a pressure in the range of 15 to 500 psig, optionally in the presence of a homogeneous or heterogeneous Gr. 8 metal catalyst, at a temperature in the range of 20 to 200° C. to obtain the mixture of 2-acetoxy carboxylic acid and 3-acetoxy carboxylic acid,
a) hydrolyzing 2-acetoxy carboxylic acid and 3-acetoxy carboxylic acid obtained in step (b) in presence of water and an acidic catalyst, at a temperature in the range of 10 to 125° C., to obtain the mixture of 2-hydroxy carboxylic acid and 3-hydroxy carboxylic acid, followed by the separation of the desired compounds of 2-hydroxy carboxylic acid and 3-hydroxycarboxylic acid from the resultant mixture by known methods and
a) reusing the catalyst, promoters and the solvent left behind after the extraction of the compounds obtained in step (a) for further cycles of hydroformylation reactions.

In an embodiment of the present invention the hydroformylation catalyst used in step (a) is a cobalt compound selected from the group consisting of cobalt chloride, cobalt bromide, cobalt iodide, cobalt acetate, cobalt nitrate, cobalt hydroxide, cobalt carbonate, cobalt sulfate, dicobalt octacarbonyl, hydridocobalt tetracarbonyl, hydridocobalt tricarbonyltributylphosphine, hydridocobalt tricarbonyltriphenyl phosphine and hydridocobalt tricarbonyltricyclohexylphosphine.

In yet another embodiment the promoter or a ligand used is an organic compound containing one or more coordinating O-atom/s selected from the group consisting of acetyl acetonate, salicylaldehyde, p-toluenesulphonic acid and an organic compound containing one or more N-atoms selected from the group consisting of pyridine, substituted pyridines, pipyridine, bipyridine, terpyridine, 1,10-phenanthroline, quinolone, isoquinoline, aniline, diphenyl amine, triphenyl amine, triethyl amine, tributyl amine, o-phenylene diamine, p-phenylene diamine, ethylene diamine and an organic compound containing coordinating N— and O-atoms selected from the group consisting of 8-hydroxy quinoline, bis (saliylidene)ethylenediamine, salicylaldoxime, picolinic acid, nicotinic acid, anthranilic acid and an organic compound containing one or more coordinating P-atom/s selected from the group consisting of trimethyl phosphine, triethyl phosphine, tri-n-butyl phosphine, tri-t-butyl phosphine, tricyclohexyl phosphine, triphenyl phosphine, bis(dicyclohexylphos phinoethane), bis(dicyclohexylphosphinobutane), bis(diphenylphosphinoethane), bis(diphenylphosphinopropane), bis(diphenyl phosphinobutane), bis(diphenylphos phinohexane) and an organic compound selected from triphenyl arsine and triphenyl stibine.

In yet another embodiment the temperature used for hydroformylation reaction in step (a) is in the range of 90 to 120° C.

In yet another embodiment the combined pressure of carbon monoxide and hydrogen used for hydroformylation reaction in step (a) is in the range of 1000 to 1600 psig.

In yet another embodiment the mole ratio of CO to H₂ in a mixture of CO and H₂ used in step (a) is in the range of 4:1 to 1:4.

In yet another embodiment the organic solvent used in step (a) is selected from the group consisting of toluene, benzene, chloroform, dichloromethane, dichloroethane, chlorobenzene, o-dichlorobenzene, p-dichlorobenzene and ketone selected from a group consisting of acetone, ethyl methyl ketone, diethyl ketone, acetophenone and cyclic ether selected from tetrahydrofuran, dioxan, and nitrile selected from acetonitrile and benzonitrile.

In yet another embodiment the products of step (a) are separated either by distillation or solvent extraction by using a solvent, which is immiscible in the hydroformylation reaction media.

In yet another embodiment the reaction mixture of step (a), after completion, is flushed with nitrogen and used as such for the oxidation of the hydroformylation products therein.

In yet another embodiment the pressure air or oxygen used for oxidation reaction in step (b) is in the range of 15 to 200 psig.

In yet another embodiment the temperature used for oxidation reaction in step (b) is in the range of 10 to 100° C.

In yet another embodiment the organic solvent used in step (b) is selected from the group consisting of toluene, benzene, chloroform, dichloromethane, dichloroethane, chlorobenzene, o-dichlorobenzene, p-dichlorobenzene and ketone selected from a group consisting of acetone, ethyl methyl ketone, diethyl ketone, acetophenone and a cyclic ether selected from tetrahydrofuran, dioxan and nitrile selected from acetonitrile and benzonitrile.

In yet another embodiment the catalyst used for the oxidation reaction in step (b) is a compound containing a Gr. 8 metal salt, Gr. 8 metal complex or Gr. 8 metal supported on a support such as carbon, silica or alumina.

In yet another embodiment the catalyst used in step (b) is separated by filtration or precipitation, after completion of the oxidation reaction and is reused for further cycle of oxidation reactions.

In yet another embodiment the products obtained in step (b) are separated by distillation or solvent extraction.

In yet another embodiment the acidic catalyst used in step (c) is selected from the group consisting of aq. HCl, aq. H₂SO₄ and organic sulfonic acid selected from p-toluene sulfonic acid and methane sulfonic acid.

In yet another embodiment the acidic catalyst used in step (c) is a heteropoly acid, a resin, selected from Amberlite IR 120 and Dowex.

In yet another embodiment the temperature used for hydrolysis reaction in step (c) is in the range of 20 to 150° C.

In yet another embodiment the catalyst used in (c), after product separation, is recycled for hydrolysis reactions in step (c).

In still another embodiment the reactions are carried is operated in a continuous manner.

### EXAMPLES

The invention is described herein bellow with reference to the following examples, which are illustrated and should not be construed as limiting the scope of the invention in any manner.

### Example 1

### Step-1

A 50 ml autoclave was charged with the following:

| | | |
|---|---|---|
| Vinyl acetate (VAM): | 3 | ml |
| Toluene: | 22 | ml |
| Co₂(CO)₈: | 0.1 | g |

The contents were flushed with nitrogen twice to ensure the removal of other gases and pressurized to 900 psig with a CO/H₂ gas mixture (mole ratio=1:1) and then heated to 120° C. The reactor pressure was maintained at 1100 psig constant by using a constant pressure regulator, which fed the CO/H₂ gas mixture from a storage reservoir. The reduction in pressure was measured from the reservoir. Reaction was monitored by absorption of gas from the reservoir. The reaction was stopped after 1 hour, when there was no depletion of pressure from the reservoir. The reactor was cooled to room temperature and the gas mixture vented off. The liquid contents were analyzed by gas chromatography. The results of the gas chromatography showed 98% conversion of VAM and selectivity to 2-acetoxy propanal and 3-acetoxy propanal was 51.37 and 48.83% respectively. The products were confirmed by GC-MS.

### Step-2

The reactor containing hydroformylation reaction mixture was flushed thrice with nitrogen and heated to 50° C., thereafter air (100 psig) was introduced into the reactor. The reactor pressure was maintained by supplying oxygen from a reservoir through a constant pressure regulator. The reaction was stopped after 2 hours, when there was no depletion of pressure from the reservoir. The reactor was cooled to room temperature and the gas was vented off. The liquid contents were analyzed by gas chromatography. The GC analysis showed 98.5% conversion of 2-acetoxypropanal with 98% selectivity to 2-acetoxypropionic acid and 100% conversion of 3-acetoxypropanal with 100% selectivity to 3-acetoxy propionic acid. The products were confirmed by GC-MS.

### Step-3

The reaction mixture after Step-2 was distilled out to separate oxidation products (2- and 3-acetoxy propionic acids) from the reaction mixture. Water (30 ml) and Amberlite IR 120 resin (0.1 g) were added to the mixture of 2- and 3-acetoxypropionic acids. The contents were heated and maintained to 80° C. for 16 hrs. The liquid contents were analyzed by gas chromatography. The results showed 99.3% conversion of 2-acetoxypropionic acid and 100% conversion of 3-acetoxy propionic acid. 2- and 3-hydroxypropionic acids were formed quantitatively, which were confirmed by GCMS.

### Example 2

A 50 ml autoclave was charged with the following:

| | | |
|---|---|---|
| Vinyl acetate (VAM): | 3 | ml |
| Toluene: | 22 | ml |
| Co₂(CO)₈: | 0.1 | g |

Hydroformylation was carried out at 1100 psig CO/H₂ gas mixture (mole ratio=1:1) and 120° C. for 1 hour.

Hydroformylation product mixture containing 2- and 3-acetoxypropanal was separated from the solvent and catalyst components by distillation. The product mixture and 22 ml toluene were charged to the reactor and oxidation (step 2) was carried out at 50° C. for 2 hours using 100-psig of air. The GC analysis after reaction showed 90% conversion of 2-acetoxypropanal with 97% selectivity to 2-acetoxypropionic acid and 95% conversion of 3-acetoxypropanal with 100% selectivity to 3-acetoxy propionic acid.

Product mixture containing 2-acetoxypropionic acid and 3-acetoxy propionic acid was separated by distillation and hydrolysis was carried out in 30 ml water at 80° C. for 16 hrs using Amberlite IR 120 resin (0.1 g). GC analysis after the reaction showed that conversion of 2-acetoxypropionic acid was 99% and conversion of 3-acetoxy propionic acid was 97.5%.

### Example 3

A 50 ml autoclave was charged with the following:

| | | |
|---|---|---|
| Vinyl acetate (VAM): | 3 | ml |
| Toluene: | 22 | ml |
| Co₂(CO)₈: | 0.1 | g |

Hydroformylation was carried out at 1100 psig CO/H₂ gas mixture (mole ratio=1:1) and 120° C. for 1 hour.

Hydroformylation product mixture containing 2- and 3-acetoxypropanal was separated from the solvent and catalyst components by distillation. The product mixture, 22 ml toluene and 5% Pd/C (0.1 g) were charged to the reactor and oxidation (step 2) was carried out at 50° C. for 2 hours using 100-psig of air. The GC analysis after reaction showed 99% conversion of 2-acetoxypropanal with 98% selectivity to 2-acetoxypropionic acid and 100% conversion of 3-acetoxypropanal with 100% selectivity to 3-acetoxypropionic acid.

Product mixture containing 2-acetoxypropionic acid and 3-acetoxy propionic acid was separated by distillation and hydrolysis was carried out in 30 ml water at 100° C. for 16 hrs using p-toluene sulfonic acid (0.1 g) as a catalyst. GC analysis after the reaction showed that conversion of 2-acetoxypropionic acid was 95% and conversion of 3-acetoxy propionic acid was 97.6%. 2- and 3-hydroxypropionic acids were formed quantitatively, which were confirmed by GCMS.

### Example 4

A 50 ml autoclave was charged with the following:

| | | |
|---|---|---|
| Vinyl acetate (VAM): | 3 | ml |
| Toluene: | 22 | ml |
| Co₂(CO)₈: | 0.1 | g |

Hydroformylation was carried out at 1100 psig CO/H₂ gas mixture (mole ratio=1:1) and 120° C. for 1 hour.

Hydroformylation product mixture containing 2- and 3-acetoxypropanal was separated from the solvent and catalyst components by distillation. The product mixture, 22 ml toluene and 5% Ru/C (0.1 g) were charged to the reactor and oxidation (step 2) was carried out at 50° C. for 25 minutes using 100-psig of air. The GC analysis after reaction showed 100% conversion of 2-acetoxypropanal with 98% selectivity to 2-acetoxypropionic acid and 100% conversion of 3-acetoxypropanal with 100% selectivity to 3-acetoxypropionic acid.

Product mixture containing 2-acetoxypropionic acid and 3-acetoxy propionic acid was separated by distillation and hydrolysis was carried out in 30 ml water at 100° C. for 16 hrs using 37% hydrolysis hydrochloric acid (1 ml) as catalyst. GC analysis after the reaction showed that conversion of 2-acetoxypropionic acid was 94.5% and conversion of 3-acetoxy propionic acid was 95.7%.

### Example 5

A 50 ml autoclave was charged with the following:

| | | |
|---|---|---|
| Vinyl acetate (VAM): | 5 | ml |
| Toluene: | 20 | ml |
| Co₂(CO)₈: | 0.1 | g |

Hydroformylation was carried out at 1100 psig CO/H₂ gas mixture (mole ratio=1:1) and 120° C. for 1 hour. The conversion of VAM was found to be 98% and selectivity to 2-acetoxypropanal and 3-acetoxypropanal was 50.49 and 46.37% respectively.

Hydroformylation product mixture containing 2- and 3-acetoxypropanal was separated from the solvent and catalyst components by distillation. The product mixture, 20 ml toluene and 5% Mn/C (0.1 g) were charged to the reactor and oxidation (step 2) was carried out at 50° C. for 25 minutes using 100-psig of air. The GC analysis after reaction showed 97.8% conversion of 2-acetoxypropanal with 98% selectivity to 2-acetoxypropionic acid and 98.3% conversion of 3-acetoxypropanal with 100% selectivity to 3-acetoxypropionic acid.

Product mixture containing 2-acetoxypropionic acid and 3-acetoxy propionic acid was separated by distillation and hydrolysis was carried out in 30 ml water at 80° C. for 16 hrs using Amberlite IR 120 resin (0.1 g) as catalyst. GC analysis after the reaction showed that conversion of 2-acetoxy propionic acid was 98.5% and conversion of 3-acetoxy propionic acid was 96.5%. 2 and 3 hydroxy propionic acids were formed quantitatively, which were confirmed by GCMS.

### Example 6

A 50 ml autoclave was charged with the following:

| | | |
|---|---|---|
| Vinyl acetate (VAM): | 10 | ml |
| Toluene: | 15 | ml |
| Co₂(CO)₈: | 0.1 | g |

Hydroformylation was carried out at 1100 psig CO/H₂ gas mixture (mole ratio=1:1) and 120° C. for 1 hour. The conversion of VAM was found to be 98% and selectivity to 2-acetoxy propanal and 3-acetoxy propanal was 51.5 and 45.37% respectively.

Hydroformylation product mixture containing 2- and 3-acetoxypropanal was separated from the solvent and catalyst components by distillation. The product mixture, 15 ml toluene and 5% Ru/C (0.1 g) were charged to the reactor and oxidation (step 2) was carried out at 50° C. for 25 minutes using 100-psig of air. The conversion of 2 and 3-acetoxy propanal was found to be 99% and 100% respectively and selectivity to 2-acetoxy propanic acid and 3-acetoxy proponic acid was 98 and 100% respectively.

Product mixture containing 2-acetoxypropionic acid and 3-acetoxy propionic acid was separated by distillation and hydrolysis was carried out in 30 ml water at 80° C. for 16 hrs using Amberlite IR 120 resin (0.1 g) as catalyst. GC analysis after the reaction showed that conversion of 2-acetoxy propionic acid was 99% and conversion of 3-acetoxy propionic acid was 97.5% with 95%. 2 and 3 hydroxy propionic acids were formed quantitatively, which were confirmed by GCMS.

### Example 7

A 50 ml autoclave was charged with the following:

| | | |
|---|---|---|
| Vinyl acetate (VAM): | 15 | ml |
| Toluene: | 10 | ml |
| Co₂(CO)₈: | 0.1 | g |

Hydroformylation was carried out at 1100 psig CO/H₂ gas mixture (mole ratio=1:1) and 120° C. for 1 hour. The conversion of VAM was found to be 97.8% and selectivity to 2-acetoxy propanal and 3-acetoxy propanal was 50.01 and 47.7% respectively.

Hydroformylation product mixture containing 2- and 3-acetoxypropanal was separated from the solvent and catalyst components by distillation. The product mixture, 10 ml toluene and 5% Ru/C (0.1 g) were charged to the reactor and oxidation (step 2) was carried out at 50° C. for 25 minutes using 100-psig of air. The conversion of 2 and 3-acetoxy propanal was found to be 98% and 98.5% respectively and selectivity to 2-acetoxy propanic acid and 3-acetoxy proponic acid was 98 and 100% respectively.

Product mixture containing 2-acetoxypropionic acid and 3-acetoxy propionic acid was separated by distillation and hydrolysis was carried out in 30 ml water at 80° C. for 16 hrs using Amberlite IR 120 resin (0.1 g) as catalyst. The conversion of 2-acetoxy propionic acid was 98.5% and conversion of 3-acetoxy propionic acid was 97.5%. 2 and 3 hydroxy propionic acids were formed quantitatively, which were confirmed by GCMS.

### Example 8

A 50 ml autoclave was charged with the following:

| | | |
|---|---|---|
| Vinyl acetate (VAM): | 20 | ml |
| Toluene: | 5 | ml |
| Co₂(CO)₈: | 0.1 | g |

Hydroformylation was carried out at 1100 psig CO/H₂ gas mixture (mole ratio=1:1) and 120° C. for 1 hour. The conversion of VAM was found to be 98% and selectivity to 2-acetoxy propanal and 3-acetoxy propanal was 50.1 and 42.7% respectively.

Hydroformylation product mixture containing 2- and 3-acetoxypropanal was separated from the solvent and catalyst components by distillation. The product mixture, 5 ml toluene and 5% Ru/C (0.1 g) were charged to the reactor and oxidation (step 2) was carried out at 50° C. for 25 minutes using 100-psig of air. The conversion of 2 and 3-acetoxy propanal was found to be 98% and 100% respectively and selectivity to 2-acetoxy propanic acid and 3-acetoxy proponic acid was 98 and 100% respectively.

Product mixture containing 2-acetoxypropionic acid and 3-acetoxy propionic acid was separated by distillation and hydrolysis was carried out in 30 ml water at 80° C. for 16 hrs using Amberlite IR 120 resin (0.1 g) as catalyst. The conversion of 2-acetoxy propionic acid was 99% and conversion of 3-acetoxy propionic acid was 97.5%. 2 and 3 hydroxy propionic acids were formed quantitatively, which were confirmed by GCMS.

### Example 9

A 50 ml autoclave was charged with the following:

| | | |
|---|---|---|
| Vinyl acetate (VAM): | 3 | ml |
| Chlorobenzene: | 22 | ml |
| Co₂(CO)₈: | 0.1 | g |

Hydroformylation was carried out at 1100 psig CO/H₂ gas mixture (mole ratio=1:1) and 120° C. for 60 min. The conversion of VAM was found to be 98% and selectivity to 2-acetoxy propanal and 3-acetoxy propanal was 41.37 and 58% respectively.

Hydroformylation product mixture containing 2- and 3-acetoxypropanal was separated from the solvent and catalyst components by distillation. The product mixture, 22 ml toluene and 5% Ru/C (0.1 g) were charged to the reactor and oxidation (step 2) was carried out at 50° C. for 25 minutes using 100-psig of air. The conversion of 2 and 3-acetoxy propanal was found to be 97% and 98.5% respectively and selectivity to 2-acetoxy propanic acid and 3-acetoxy proponic acid was 98 and 100% respectively.

Product mixture containing 2-acetoxypropionic acid and 3-acetoxy propionic acid was separated by distillation and hydrolysis was carried out in 30 ml water at 80° C. for 16 hrs using Amberlite IR 120 resin (0.1 g) as catalyst. The conversion of 2-acetoxy propionic acid was 99.3% and conversion of 3-acetoxy propionic acid was 96.5%. 2 and 3 hydroxy propionic acids were formed quantitatively, which were confirmed by GCMS.

### Example 10

A 50 ml autoclave was charged with the following:

| | | |
|---|---|---|
| Vinyl acetate (VAM): | 3 | ml |
| Dichloromethane: | 22 | ml |
| Co₂(CO)₈: | 0.1 | g |

Hydroformylation was carried out at 1100 psig CO/H₂ gas mixture (mole ratio=1:1) and 120° C. for 60 min. The conversion of VAM was found to be 98% and selectivity to 2-acetoxy propanal and 3-acetoxy propanal was 44.37 and 55% respectively.

Hydroformylation product mixture containing 2- and 3-acetoxypropanal was separated from the solvent and catalyst components by distillation. The product mixture, 22 ml toluene and 5% Ru/C (0.1 g) were charged to the reactor and oxidation (step 2) was carried out at 50° C. for 25 minutes using 100-psig of air. The conversion of 2 and 3-acetoxy propanal was found to be 98% and 99.3% respectively and selectivity to 2-acetoxy propanic acid and 3-acetoxy proponic acid was 98 and 100% respectively.

Product mixture containing 2-acetoxypropionic acid and 3-acetoxy propionic acid was separated by distillation and hydrolysis was carried out in 30 ml water at 80° C. for 16 hrs using Amberlite IR 120 resin (0.1 g) as catalyst. The conversion of 2-acetoxy propionic acid was 99.3% conversion of 3-acetoxy propionic acid was 97.5% with 95%. 2 and 3 hydroxy propionic acids were formed quantitatively, which were confirmed by GCMS.

### Example 11

A 50 ml autoclave was charged with the following:

| | | |
|---|---|---|
| Vinyl acetate (VAM): | 3 | ml |
| Toluene: | 22 | ml |
| Co₂(CO)₈: | 0.1 | g |
| Cyclooctadiene: | 0.19 | g |

Hydroformylation was carried out at 1100 psig CO/H₂ gas mixture (mole ratio=1:1) and 120° C. for 60 min. The conversion of VAM was found to be 98% and selectivity to 2-acetoxy propanal and 3-acetoxy propanal was 51 and 48% respectively.

Hydroformylation product mixture containing 2- and 3-acetoxypropanal was separated from the solvent and catalyst components by distillation. The product mixture, 22 ml toluene and 5% Ru/C (0.1 g) were charged to the reactor and oxidation (step 2) was carried out at 50° C. for 25 minutes using 100-psig of air. The GC analysis after reaction showed 98% conversion of 2-acetoxypropanal with 98% selectivity to 2-acetoxypropionic acid and 100% conversion of 3-acetoxypropanal with 100% selectivity to 3-acetoxypropionic acid.

Product mixture containing 2-acetoxypropionic acid and 3-acetoxy propionic acid was separated by distillation and hydrolysis was carried out in 30 ml water at 80° C. for 16 hrs using Amberlite IR 120 resin (0.1 g) as catalyst. The conversion of 2-acetoxy propionic acid was 99% conversion of 3-acetoxy propionic acid was 97.5%. 2 and 3 hydroxy propionic acids were formed quantitatively, which were confirmed by GCMS.

### Example 12

A 50 ml autoclave was charged with the following:

| | | |
|---|---|---|
| Vinyl acetate (VAM): | 3 | ml |
| Toluene: | 22 | ml |
| Co₂(CO)₈: | 0.1 | g |
| Pyridine: | 0.037 | g |

Hydroformylation was carried out at 1100 psig CO/H₂ gas mixture (mole ratio=1:1) and 120° C. for 45 min. The conversion of VAM was found to be 98% and selectivity to 2-acetoxy propanal and 3-acetoxy propanal was 51.8 and 42.3% respectively.

Hydroformylation product mixture containing 2- and 3-acetoxypropanal was separated from the solvent and catalyst components by distillation. The product mixture, 22 ml toluene and 5% Ru/C (0.1 g) were charged to the reactor and oxidation (step 2) was carried out at 50° C. for 25 minutes using 100-psig of air. The GC analysis after reaction showed 98.5% conversion of 2-acetoxypropanal with 98% selectivity to 2-acetoxypropionic acid and 98% conversion of 3-acetoxypropanal with 100% selectivity to 3-acetoxypropionic acid.

Product mixture containing 2-acetoxypropionic acid and 3-acetoxy propionic acid was separated by distillation and hydrolysis was carried out in 30 ml water at 80° C. for 16 hrs using Amberlite IR 120 resin (0.1 g) as catalyst. The conversion of 2-acetoxy propionic acid was 98.7% and conversion of 3-acetoxy propionic acid was 98.5%. 2 and 3 hydroxy propionic acids were formed quantitatively, which were confirmed by GCMS.

### Examples 13-18

A 50 ml autoclave was charged with the following:

| | | |
|---|---|---|
| Vinyl acetate: | 3 | ml |
| Toluene: | 22 | ml |
| Co₂(CO)₈: | 0.1 | g |

Hydroformylation was carried out at 1100 psig CO/H₂ gas mixture (mole ratio 1:1) and 120° C. for 1 hour. The gas was vented off and immediately 5 ml of water was added to reactor and it was pressurized to 400 psi of CO/H₂ gas mixture (mole ratio=1:1), to avoid the deactivation of catalyst, under vigorous stirring for half an hour. This procedure was repeated for 3 times to ensure the complete extraction of product in water phase. Thus the product phase was extracted in water and organic phase, which includes catalyst, was recycled by adding 3 ml VAM to the reactor and carrying out hydroformylation at 1100 psig CO/H₂ gas mixture (mole ratio=1:1) and 120° C. for 1 hour. This procedure was repeated for 5 times activity and selectivity was tabulated for each recycle.

| | | |
|---|---|---|
| Example | Conversion | Selectivity % |

| No | Recycle no | % | 2-acpal | 3-acpal |
|---|---|---|---|---|
| 13 | Virgin run | 99.5 | 51.37 | 47.8 |
| 14 | Recycle 1 | 98 | 51 | 48 |
| 15 | Recycle 2 | 97.8 | 51.5 | 48.3 |
| 16 | Recycle 3 | 100 | 50 | 47.5 |
| 17 | Recycle 4 | 99.3 | 49 | 46 |
| 18 | Recycle 5 | 99.1 | 51.2 | 47.8 |

### THE MAIN ADVANTAGES OF THE PRESENT INVENTION ARE

1. The process of the invention provides an efficient method for preparation of 2-hydroxy carboxylic acid and 3-hydroxy carboxylic acid.

2. The process of the present invention can be applied to prepare any intermediate compound such as 2-acetoxycarboxyaldehyde, 2-acetoxycarboxylic acid, 2-hydroxy carboxylic acid, 3-acetoxycarboxyaldehyde, 3-acetoxycarboxylic acid, 3-hydroxy carboxylic acid.

3. The process can be optimized to achieve maximum yield of any required intermediate component by selecting a combination of catalyst, promoter and a set of conditions.

4. The process uses cheaper and recyclable catalysts for all of the steps.

1. A process for the preparation of hydroxy carboxylic acids comprising the steps of:
a) hydroformylating an enol acylate having formula R₂C═C(R)—O-Acyl, wherein R is H, alkyl or aryl group containing 1 to 15 carbon atoms free from any ethylenic or acetylenic unsaturation, and each R is the same or different, with a mixture of carbon monoxide and hydrogen, at a pressure in the range of 500 to 5000 psig, at a temperature in the range of 50 to 140° C., in presence of a catalyst containing cobalt, optionally in the presence of a promoter or a ligand containing O, N, N—O, P, As or Sb atom/s and an organic solvent to obtain a mixture of 2-acetoxy carboxaldehyde and 3-acetoxy carboxaldehyde,
b) oxidizing 2-acetoxy carboxaldehyde and 3-acetoxy carboxaldehyde either after their separation from the above said reaction mixture obtained in step (a) by distillation or solvent extraction, or as such in the reaction mixture without separation, in presence of air or O₂, optionally in the presence of an organic solvent, at a pressure in the range of 15 to 500 psig, optionally in the presence of a homogeneous or heterogeneous Gr. 8 metal catalyst, at a temperature in the range of 20 to 200° C. to obtain the mixture of 2-acetoxy carboxylic acid and 3-acetoxy carboxylic acid,
c) hydrolyzing 2-acetoxy carboxylic acid and 3-acetoxy carboxylic acid obtained in step (b) in presence of water and an acidic catalyst, at a temperature in the range of 10 to 125° C., to obtain the mixture of 2-hydroxy carboxylic acid and 3-hydroxy carboxylic acid, followed by the separation of the desired compounds of 2-hydroxy carboxylic acid and 3-hydroxycarboxylic acid from the resultant product mixture by known methods and
d) reusing the catalyst, promoters and the solvent left behind after the extraction of the compounds obtained in step (a) for further cycles of hydroformylation reactions.

2. The process of claim 1, wherein the hydroformylation catalyst used in step (a) is a cobalt compound selected from the group consisting of cobalt chloride, cobalt bromide, cobalt iodide, cobalt acetate, cobalt nitrate, cobalt hydroxide, cobalt carbonate, cobalt sulfate, dicobalt octacarbonyl, hydridocobalt tetracarbonyl, hydridocobalt tricarbonyltributylphosphine, hydridocobalt tricarbonyltriphenyl phosphine and hydridocobalt tricarbonyltricyclohexylphosphine.

3. The process of claim 1, wherein a promoter or a ligand used is an organic compound containing one or more coordinating O-atom/s selected from the group consisting of acetyl acetonate, salicylaldehyde, p-toluenesulphonic acid and an organic compound containing one or more N-atoms selected from the group consisting of pyridine, substituted pyridines, pipyridine, bipyridine, terpyridine, 1,10-phenanthroline, quinolone, isoquinoline, aniline, diphenyl amine, triphenyl amine, triethyl amine, tributyl amine, o-phenylene diamine, p-phenylene diamine, ethylene diamine and an organic compound containing coordinating N— and O-atoms selected from the group consisting of 8-hydroxy quinoline, bis(saliylidene)ethylenediamine, salicylaldoxime, picolinic acid, nicotinic acid, anthranilic acid and an organic compound containing one or more coordinating P-atom/s selected from the group consisting of trimethyl phosphine, triethyl phosphine, tri-n-butyl phosphine, tri-t-butyl phosphine, tricyclohexyl phosphine, triphenyl phosphine, bis(dicyclohexylphosphinoethane), bis(dicyclohexylphosphinobutane), bis(diphenylphosphinoethane), bis(diphenylphosphinopropane), bis(diphenyl phosphinobutane), bis(diphenylphosphinohexane) and an organic compound selected from triphenyl arsine and triphenyl stibine.

4. The process of claim 1, wherein the temperature used for hydroformylation reaction in step (a) is in the range of 90 to 120° C.

5. The process of claim 1, wherein the combined pressure of carbon monoxide and hydrogen used for hydroformylation reaction in step (a) is in the range of 1000 to 1600 psig.

6. The process of claim 1, wherein the mole ratio of CO to H₂ in a mixture of CO and H₂ used in step (a) is in the range of 4:1 to 1:4.

7. The process of claim 1, wherein the organic solvent used in step (a) is selected from the group consisting of toluene, benzene, chloroform, dichloromethane, dichloroethane, chlorobenzene, o-dichlorobenzene, p-dichlorobenzene and ketone selected from a group consisting of acetone, ethyl methyl ketone, diethyl ketone, acetophenone and cyclic ether selected from tetrahydrofuran, dioxan, and nitrile selected from acetonitrile and benzonitrile.

8. The process of claim 1, where the products of step (a) are separated either by distillation or solvent extraction by using a solvent, which is immiscible in the hydroformylation reaction media.

9. The process of claim 1, wherein the reaction mixture of step (a), after completion, is flushed with nitrogen and used as such for the oxidation of the hydroformylation products therein.

10. The process of claim 1, wherein the pressure air or oxygen used for oxidation reaction in step (b) is in the range of 15 to 200 psig.

11. The process of claim 1, wherein the temperature used for oxidation reaction in step (b) is in the range of 10 to 100° C.

12. The process of claim 1, wherein the organic solvent used in step (b) is selected from the group consisting of toluene, benzene, chloroform, dichloromethane, dichloroethane, chlorobenzene, o-dichlorobenzene, p-dichlorobenzene and ketone selected from a group consisting of acetone, ethyl methyl ketone, diethyl ketone, acetophenone and a cyclic ether selected from tetrahydrofuran, dioxan and nitrile selected from acetonitrile and benzonitrile.

13. The process of claim 1, wherein the catalyst used for the oxidation reaction in step (b) is a compound containing a Gr. 8 metal salt, Gr. 8 metal complex or Gr. 8 metal supported on a support such as carbon, silica or alumina.

14. The process of claim 1, where the catalyst used in step (b) is separated by filtration or precipitation, after completion of the oxidation reaction and is reused for further oxidation step.

15. The process of claim 1, wherein the products obtained in step (b) are separated by distillation or solvent extraction.

16. The process of claim 1, wherein the acidic catalyst used in step (c) is selected from the group consisting of aq. HCl, aq. H₂SO₄ and organic sulfonic acid selected from p-toluene sulfonic acid and methane sulfonic acid.

17. The process of claim 1, wherein the acidic catalyst used in step (c) is a heteropoly acid, a resin, selected from Themberlite IR 120 and Dowex.

18. The process of claim 1, wherein the temperature used for hydrolysis reaction in step (c) is in the range of 20 to 150° C.

19. The process of claim 1, where the catalyst used in (c), after product separation, is recycled for hydrolysis reactions in step (c).

20. The process of claim 1, wherein the reactions are carried is operated in a continuous manner.
